(19) United States
(12) Patent Application Publication
(10) Pub. No.: US 2025/0332037 A1
(43) Pub. Date: Oct. 30, 2025

(54) ALL IN ONE DISPOSABLE INCONTINENCE/MOISTURE CAPTURE

(71) Applicant: Elmroi LLC, Addison, TX (US)

(72) Inventor: Akinwale Joseph Ibiloye, Addison, TX (US)

(73) Assignee: Elmroi LLC, Addison, TX (US)

(21) Appl. No.: 19/186,114

(22) Filed: Apr. 22, 2025

Related U.S. Application Data
(60) Provisional application No. 63/638,104, filed on Apr. 24, 2024.

(51) Int. Cl. A61F 13/45; A61F 13/15

(52) U.S. Cl. CPC A61F 13/45; A61F 13/15707; A61F 2013/15715; A61F 2013/4506; A61F 2013/4518

(57) ABSTRACT

A personal hygiene device comprising an outer shell configured to be disposed on a patient. An inner absorbent material disposed interior to the outer shell and forming a single unit with the outer shell. A tube having a first end and a second end disposed interior to the inner absorbent material, wherein the first end of the tube is configured to remove a liquid from the absorbent material when suction is applied to the second end.

### RELATED APPLICATIONS

The present application claims benefit of and priority to U.S. Provisional Patent Application no. 63/638, 104, filed Apr. 24, 2024, which is hereby incorporated by reference for all purposes as if set forth herein in its entirety.

### TECHNICAL FIELD

The present disclosure relates generally to personal care products, and more specifically to an all in one disposable incontinence/moisture capture device and a method of manufacture.

### BACKGROUND OF THE INVENTION

Disposable incontinence devices are widely used and often lead to skin irritation or worse. The usual way to deal with such problems is application of topical skin irritation treatment ointments, which fail to prevent skin irritation.

### SUMMARY OF THE INVENTION

A personal hygiene device is disclosed that includes an outer shell configured to be disposed on a patient, such as a waterproof article of apparel that forms a moisture-proof seal against the patient's body. An inner absorbent material is disposed interior to the outer shell and forms a single unit with the outer shell. A tube having a first end and a second end is disposed interior to the inner absorbent material, wherein the first end of the tube is configured to remove a liquid from the absorbent material when suction is applied to the second end.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a device for all in one disposable incontinence and moisture capture, in accordance with an example embodiment of the present disclosure;

FIG. 2 is a diagram of a device for all in one disposable incontinence and moisture capture with additional padding, in accordance with an example embodiment of the present disclosure;

FIG. 3 is a diagram of a device for all in one disposable incontinence and moisture capture with an extended suction field, in accordance with an example embodiment of the present disclosure; and FIG. 4 is a diagram of a process for manufacturing and using an all in one disposable incontinence and moisture capture device, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

The present disclosure provides a device and method of operation that provides protection from moisture associated skin damage (MASD), along with incontinence associated dermatitis (IAD) prevention. In particular, the present disclosure recognizes that prompt removal of fluids can significantly increase the rate of successful outcomes, and that existing devices fail to do so for a number of reasons that are readily apparent, including the fact that they are separate from protective garments, such as disposable protective garments. Combining moisture removal with disposable protective garments results in a slight increase in cost, but by preventing MASD and IAD, an overall cost savings is realized.

The present application claims benefit of and priority to U.S. Provisional Patent Application no. 63/638,104, filed Apr. 24, 2024, which is hereby incorporated by reference for all purposes as if set forth herein in its entirety.

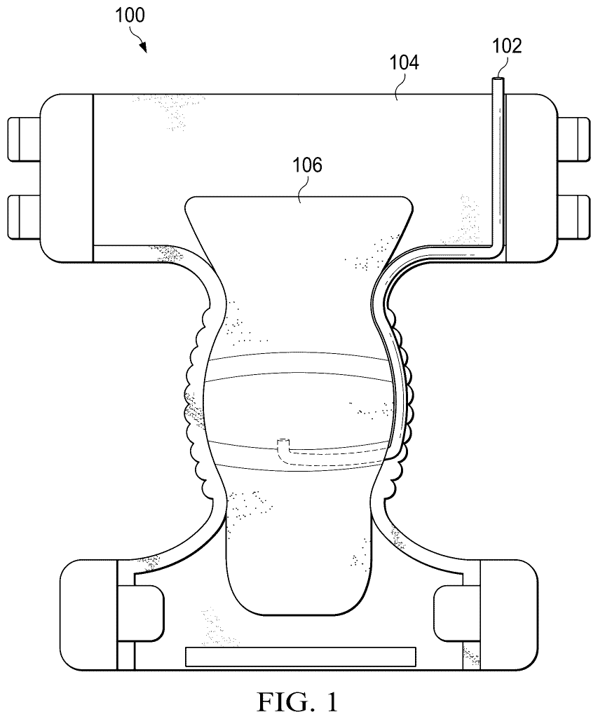

FIG. 1 is a diagram of a device 100 for all in one disposable incontinence and moisture capture, in accordance with an example embodiment of the present disclosure. Device 100 can be a personal hygiene device that is implemented in disposable materials, reusable materials, reusable components with disposable parts or in other suitable manners.

Device 100 includes suction tube 102 which is disposed inside of outer shell 104 and within absorbent material 106. In one example embodiment, suction tube 102 can have a length L with a first end and a second end and can be fabricated from a flexible, crush-proof disposable tubing that is configured to be connected to a suction device. One end of suction tube 102 can extend exterior to outer shell 104, and a second end of suction tube 102 can be disposed within absorbent material 106, in a location where fluids typically accumulate. For example, a patient who is confined to a bed might require a device 100 that is configured for fluid accumulation towards the rear of device 100, whereas a patient who is confined to a wheel chair might require a device 100 that is configured for fluid accumulation towards the bottom of device 100. In this manner, device 100 can be configured for specific patients and applications. Suction tube 102 can include a filter or other suitable devices that prevent solid materials from blocking moisture removal.

Outer shell 104 can be fabricated from a water proof plastic material that can contain moisture, so as to allow suction tube 102 to remove moisture and to prevent moisture from leaking out of outer shell 104 when it is being worn by a patient. Outer shell 104 can have a plurality of adhesive tabs 108 disposed at locations to aid with providing a tight fit for device 100 on a patient. Outer shell 104 can be disposable, can be configured for absorbent material 106 to be removed and replaced so as to be reusable or can have other suitable configurations. In another example embodiment, device 100 can be configured with a continuous structure in a pants-like fashion that uses elastic bands or other elastic features to hold outer shell 104 against the patient's body, so as to allow device 100 to be worn by a patient without the need for adhesive tabs 108 or other assembly.

Absorbent material 106 can be fabricated from cotton, hemp, bamboo microfiber, a moisture wicking poly blend material or other suitable materials that can absorb and retain large volumes of liquids. In one example embodiment, absorbent material 106 can include one or more odor absorbing components, such as activated charcoal, silica gel or other suitable materials that can help to control offensive odors from liquids contained within device 100. In another example embodiment, absorbent material 106 can be removable, so as to allow it to be replaced within outer shell 104.

In operation, device 100 can be used to remove moisture from a patient who is incontinent or otherwise unable to use sanitary waste disposal facilities. Device 100 includes a suction tube 102 that can be attached to a suction device that is configured to remove moisture, liquids or solids from the interior of device 100.

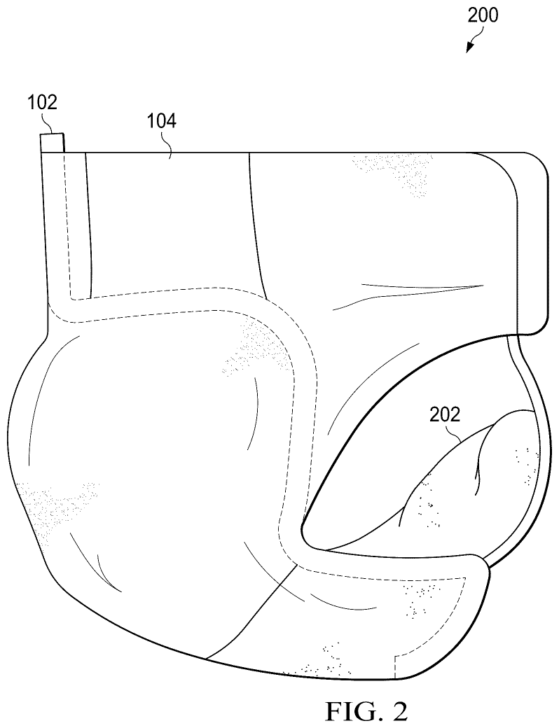

FIG. 2 is a diagram of a device 200 for all in one disposable incontinence and moisture capture with additional padding, in accordance with an example embodiment of the present disclosure. Device 200 can be implemented in disposable materials, reusable materials, reusable components with disposable parts or in other suitable manners. Device 200 includes padding 202, which is disposed over areas that are susceptible to injury, such as bone that is not well protected by muscle and fat, tissue that is subject to abrasion or irritation, or other areas where additional padding is of benefit to a patient. In one example embodiment, padding 202 can be removable, such as using adhesives, Velcro or other suitable mechanisms. Padding 202 can comprise a combination of an antimicrobial polyurethane foam and cotton or other suitable materials.

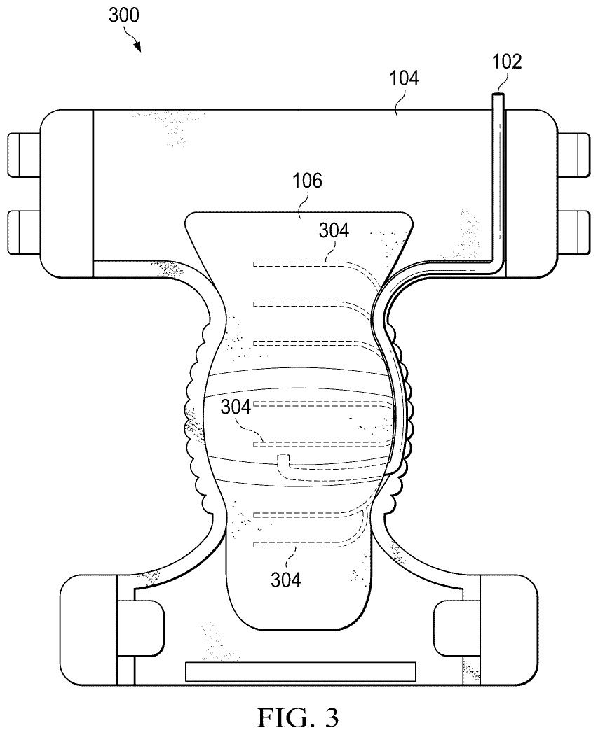

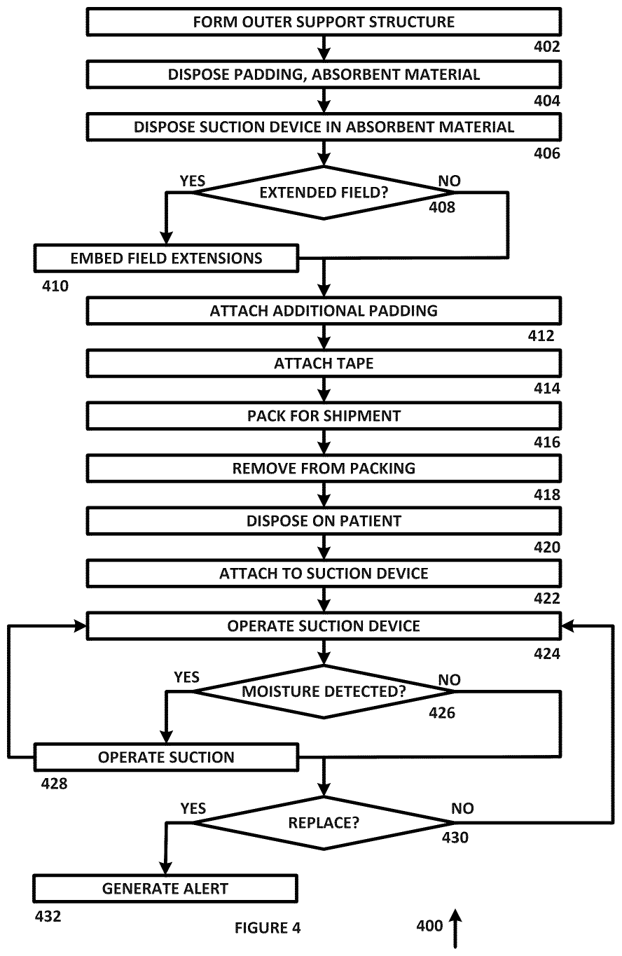

FIG. 3 is a diagram of a device 300 for all in one disposable incontinence and moisture capture with an extended suction field, in accordance with an example embodiment of the present disclosure. Device 300 can be implemented in disposable materials, reusable materials, reusable components with disposable parts or in other suitable manners.

Device 300 is similar to device 100, except that it includes suction field 304. In one example embodiment, suction field 304 can be implemented using a plurality of suction tubes that are connected to suction tube 102 through a manifold and distributed in areas within outer shell 104 where fluid is expected to accumulate, such as within all of outer shell 104, in predetermined locations within outer shell 104 or in other suitable manners. In another example embodiment, suction field 304 can be fabricated from layers of material with channels disposed within the layers and breather fabric, filter devices or other suitable materials that are able to distribute the suction field.

Having an increased amount of capillary tubes along with the size of the holes in the tubes can help to prevent clogging. The flow of suction from the main tube can remain constant, so if a capillary tube gets clogged then the others will have increased suction, which will help prevent further clogging. The suction resistance can be monitored to generate an objective indication of clogging. An alert or notification can be generated if a predetermined percentage of the capillary tubes are not working properly, such as from 30% to 70%.

FIG. 4 is a diagram of a process 400 for manufacturing and using an all in one disposable incontinence and moisture capture personal hygiene device, in accordance with an example embodiment of the present disclosure. The manufacturing elements of process 400 can be implemented by automated manufacturing equipment or in other suitable manners, and the use elements of process 400 can be implemented using automated pumping and monitoring equipment or in other suitable manners.

Process 400 begins at 402, where an outer support structure is formed. In one example embodiment, the outer support structure can be cut or punched from a stack of plastic sheet layers by automated manufacturing equipment operating under control of a programmable controller, can be extruded, printed or formed in other suitable manners. The process then proceeds to 404.

At 404, padding and absorbent material is disposed. In one example embodiment, padding can be attached across an outer surface area of the support structure, including where the device attaches together with tape. The absorbent material can be attached on an inner surface of the outer support structure. In one example embodiment, the absorbent material can be fabricated from cotton, hemp, bamboo microfiber or other suitable materials that can absorb and retain large volumes of liquids, and can be disposed by bonding with epoxy or other suitable adhesives or in other suitable manners. The process then proceeds to 406.

At 406, a suction device is disposed in the absorbent material. In one example embodiment, the suction device can be a tube with an attachment on one end for connection to a vacuum device or other suitable structures, and a filter on the other end or other suitable structures. The suction device can be stitched into the absorbent material, attached with epoxy or other suitable adhesives or attached in other suitable manners. In another example embodiment, the suction device can be disposed on the inner surface of the support structure prior to attachment of the absorbent material, or other suitable processes can also or alternatively be used. The process then proceeds to 408.

At 408, it is determined whether an extended suction field is to be provided. If it is determined that an extended suction field does not need to be provided, the process proceeds to 412, otherwise the process proceeds to 410 where the suction field extension is embedded. In one example embodiment, a manifold can be attached to the suction tube that distributes the suction in an extended area of the absorbent material, such as the bottom, the back or in other suitable locations. Likewise, the extended area can include the entire inner surface of the absorbent material. For example, layers of material with channels disposed within the layers and breather fabric, filter devices or other suitable materials that are able to distribute the suction field can be used. The process then proceeds to 412.

At 412, any additional padding is attached to the outer surface of the support structure. In one example embodiment, the patient may have bone or other structures that are subject to being irritated, and additional padding can be provided to protect such sensitive areas from injury or irritation. The process then proceeds to 414.

At 414, tape can be attached to the support structure. In one example embodiment, tape can be used to secure the support structure on a patient, and can be attached with removable covers to facilitate placement of the device on a patient. The device can also be designed with elastic components and a continuous construction around the waist and legs, so as to avoid the need for tape or in other suitable manners. The process then proceeds to 416.

At 416, the personal hygiene device is packed for shipment. In one example embodiment, the personal hygiene device can be packed according to a classification of the personal hygiene device (e.g. sitting or prone), a type of suction field (e.g. localized or distributed) or based on other characteristics. The process then proceeds to 418.

At 418, the personal hygiene device is removed from packaging. In one example embodiment, the device can be removed in a manner that facilitates use. The process then proceeds to 420.

At 420, the personal hygiene device is disposed on a patient. In one example embodiment, the processes discussed and described herein can be used or other suitable processes can also or alternatively be used. The process then proceeds to 422.

At 422, the personal hygiene device is attached to a suction generating device. In one example embodiment, the suction generating device can be configured to operate with a product having the unique configuration of the device, such as the ability to run continuously as opposed to be periodically switched on or off by a user. The process then proceeds to 424.

At 424, the suction generating device is operated. The suction generating device can be a wet-dry suction device that can tolerate moisture, that is configured to generate suction pressure at a designed level based on a design of the personal hygiene device, and to monitor the suction pressure or other suitable metrics to determine a state of health of the personal hygiene device. In one example embodiment, the suction generating device can have a standby mode where a periodic check for moisture is performed, to prevent the device from constantly providing suction and causing patient discomfort or other problems. The process then proceeds to 418.

At 426, it is determined whether moisture is present. In one example embodiment, the determination can be made with a sensor that detects moisture above a predetermined level or in other suitable manners. If it is determined that moisture is present, the process proceeds to 428 where suction is generated to remove the moisture and then stopped when moisture falls below the predetermined level or based on other measured parameters, and then return to 424. Otherwise, the process proceeds to 430.

At 430, it is determined whether the personal hygiene device needs to be replaced. In one example embodiment, the personal hygiene device can be replaced after a predetermined period of time, after a predetermined number of vacuum cycles, or in other suitable manners. If it is determined that the personal hygiene device needs to be replaced, the algorithm proceeds to 432 and an alert or notification is generated, such as to notify the patient or caregiver of the need to change the personal hygiene device. Otherwise the algorithm returns to 424.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A personal hygiene device comprising:
an outer shell configured to be disposed on a patient;
an inner absorbent material disposed interior to the outer shell and forming a single unit with the outer shell; and
a tube having a first end and a second end disposed interior to the inner absorbent material, wherein the first end of the tube is configured to remove a liquid from the absorbent material when suction is applied to the second end.

2. The personal hygiene device of claim 1 further comprising:
a manifold coupled to the first end of the tube; and
a plurality of tubes extending from the manifold to form a distributed suction field.

3. The personal hygiene device of claim 1 further comprising:
a manifold coupled to the first end of the tube; and
a structure extending from the manifold forming a distributed suction field.

4. The personal hygiene device of claim 1 wherein the tube is disposed between the inner absorbent material and the outer shell.

5. The personal hygiene device of claim 1 further comprising padding disposed on an external surface of the outer shell.

6. The personal hygiene device of claim 1 further comprising a suction device coupled to the tube, the suction device configured to detect when a suction pressure has dropped below a predetermined level and to generate an alert.

7. The personal hygiene device of claim 1 wherein the outer shell comprises a continuous construction suitable for use without adhesive tabs.

8. The personal hygiene device of claim 1 wherein the outer shell comprises a plurality of adhesive tabs.

9. The personal hygiene device of claim 1 wherein the tube comprises a plurality of holes disposed along a length of the tube.

10. A method of manufacturing a personal hygiene device comprising:
forming an outer shell configured to be disposed on a patient;
disposing an inner absorbent material interior to the outer shell to form a single unit with the outer shell; and
disposing a tube having a first end and a second end interior to the inner absorbent material, wherein the first end of the tube is configured to remove a liquid from the absorbent material when suction is applied to the second end.

11. The method of claim 10 further comprising:
forming a manifold coupled to the first end of the tube; and
forming a plurality of tubes extending from the manifold to create a distributed suction field.

12. The method of claim 10 further comprising:
forming a manifold coupled to the first end of the tube; and
forming a structure extending from the manifold to create a distributed suction field.

13. The method of claim 10 wherein the tube is disposed between the inner absorbent material and the outer shell.

14. The method of claim 10 further comprising disposing padding on an external surface of the outer shell.

15. The method of claim 10 further comprising coupling a suction device to the tube, the suction device configured to detect when a suction pressure has dropped below a predetermined level and to generate an alert.

16. The method of claim 10 wherein the outer shell comprises a continuous construction suitable for use without adhesive tabs.

17. The method of claim 10 wherein the outer shell comprises a plurality of adhesive tabs.

18. The method of claim 10 wherein the tube comprises a plurality of holes disposed along a length of the tube.

19. A method of using a disposable personal hygiene device, comprising:
disposing the disposable personal hygiene device on a user;
connecting a suction tube of the personal hygiene device to a suction device; and
generating a notification when a suction power level has dropped below a predetermined level.

20. The method of claim 19 wherein the suction device is a wet suction device.
